SEQUENCE LISTING (10 sequences)

SEQ ID NO: 1 - DNA, 3252 bp, synthetic construct
  source 1..3252: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctcctttggaggtggcggttctggcggaggtggaagtaccttttctaagctgagagaacagctgggccccgtgacacaagagttctgggacaacctggaaaaagagacagagggcctgagacaagagatgagcaaggacctggaagaagtgaaggccaaggtccagccttacctggacgacttccagaagaaatggcaagaggaaatggaactgtaccggcagaaggtggaacccctgagagccgagcttcaagaaggcgccagacagaagctgcacgagctgcaagagaagctgagccctctgggagaagagatgcgggacagagctagagcccatgtggatgccctgagaacccatctggccccatacctggatgattttcagaaaaagtggcaagaagagatggaactctatcgccagaaagtcgagcctctgcgggccgaattgcaagagggtgctagacagaaactccatgaactccaagagaaactgtccccactgggcgaagaaatgagagatagagccagggctcacgtggacgccctcaggacacatctcgccccttactccgatgagctgcggcaaagactggccgccagactggaagccctgaaagaaaatggcggcgctaggctggccgagtatcacgccaaagccacagagcacctgagcaccctgagcgagaaagccaaacctgctctggaagatctgcggcagggactgctgcctgtgctggaaagcttcaaggtgtccttcctgagcgccctggaagagtacaccaagaagctgaacacccagggtggcggcggaagccaccaccatcatcaccaccatcaccatcac

SEQ ID NO: 2 - protein, 1084 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSFGGGGSGGGGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGGGGSHHHHHHHHHH

SEQ ID NO: 3 - protein, 21 aa, Homo sapiens
IWLIVFGVVMGVIVVGIVILI

SEQ ID NO: 4 - DNA, 3054 bp, synthetic construct
  source 1..3054: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccggcccgggaggcggcggaggtggaagtaccttttctaagctgagagaacagctgggccccgtgacacaagagttctgggacaacctggaaaaagagacagagggcctgagacaagagatgagcaaggacctggaagaagtgaaggccaaggtccagccttacctggacgacttccagaagaaatggcaagaggaaatggaactgtaccggcagaaggtggaacccctgagagccgagcttcaagaaggcgccagacagaagctgcacgagctgcaagagaagctgagccctctgggagaagagatgcgggacagagctagagcccatgtggatgccctgagaacccatctggccccatacctggatgattttcagaaaaagtggcaagaagagatggaactctatcgccagaaagtcgagcctctgcgggccgaattgcaagagggtgctagacagaaactccatgaactccaagagaaactgtccccactgggcgaagaaatgagagatagagccagggctcacgtggacgccctcaggacacatctcgccccttactccgatgagctgcggcaaagactggccgccagactggaagccctgaaagaaaatggcggcgctaggctggccgagtatcacgccaaagccacagagcacctgagcaccctgagcgagaaagccaaacctgctctggaagatctgcggcagggactgctgcctgtgctggaaagcttcaaggtgtccttcctgagcgccctggaagagtacaccaagaagctgaacacccagggtggcggcggaagccaccaccatcatcaccaccatcaccatcac

SEQ ID NO: 5 - protein, 1018 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGPGGGGGGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGGGGSHHHHHHHHHH

SEQ ID NO: 6 - DNA, 2460 bp, Homo sapiens
  source 1..2460: mol_type = genomic DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctcctttgggggtggaggctctcaccatcaccaccatcatcaccaccatcac

SEQ ID NO: 7 - protein, 820 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSFGGGGSHHHHHHHHHH

SEQ ID NO: 8 - DNA, 2265 bp, synthetic construct
  source 1..2265: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccgggggtggaggctctcaccatcaccaccatcatcaccaccatcac

SEQ ID NO: 9 - protein, 755 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSHHHHHHHHHH

SEQ ID NO: 10 - DNA, 1521 bp, Homo sapiens
  source 1..1521: mol_type = genomic DNA
atggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggcgggggtggaggctctcaccatcaccaccatcatcaccaccatcac

(19) United States
(12) Patent Application Publication
(10) Pub. No.: US 2025/0152681 A1
(43) Pub. Date: May 15, 2025

(54) NANODISC COMPRISING ANGIOTENSIN CONVERTING ENZYME 2

(71) Applicants: RESEARCH & BUSINESS FOUNDATION SUNGKYUNKWAN UNIVERSITY, Suwon-si, Gyeonggi-do (KR); MVRIX Co.,Ltd., Hwaseong-si, Gyeonggi-do (KR)

(72) Inventors: Dae Hyuk KWEON, Seoul (KR); Jae Hyeon HWANG, Suwon-si (KR); Kyeong Won KIM, Suwon-si (KR); Jeong Hyeon YOON, Suwon-si (KR); Won Beom PARK, Suwon-si (KR)

(73) Assignees: RESEARCH & BUSINESS FOUNDATION SUNGKYUNKWAN UNIVERSITY, Suwon-si, Gyeonggi-do (KR); MVRIX Co.,Ltd., Hwaseong-si, Gyeonggi-do (KR)

(21) Appl. No.: 18/839,970

(22) PCT Filed: Feb. 10, 2023
(86) PCT No.: PCT/KR2023/001967

§ 371 Date: Aug. 20, 2024

(30) Foreign Application Priority Data
Feb. 21, 2022 (KR) 10-2022-0022501
Feb. 7, 2023 (KR) 10-2023-0016367

(51) Int. Cl. A61K 38/48; A61K 47/69; A61P 31/12

(52) U.S. Cl. CPC A61K 38/4813; A61K 47/6911; A61P 31/12; C12Y 304/17023

(57) ABSTRACT

The present invention relates to a nanodisc comprising angiotensin converting enzyme 2 (ACE2) and a membrane scaffold protein (MSP). In the present invention, the angiotensin converting enzyme 2 (ACE2) is included in the nanodisc, and it was confirmed that the antiviral efficacy and substrate conversion ability of the angiotensin converting enzyme 2 (ACE2) could be significantly improved.

### TECHNICAL FIELD

The present invention relates to a nanodisc containing angiotensin converting enzyme 2 (ACE2) and a membrane scaffold protein (MSP) polymer.

### BACKGROUND ART

Angiotensin-converting enzyme 2 (ACE2) plays an important role in the renin-angiotensin-aldosterone system (RAAS), which regulates body moisture and blood pressure.

In the renin-angiotensin-aldosterone system (RAAS), renin converts angiotensinogen into angiotensin I, and angiotensin converting enzyme (ACE) converts angiotensin I into angiotensin II. Angiotensin II has a direct adverse effect on the cardiovascular system, for example, causes inflammatory reactions within blood vessels and promotes atherosclerosis. However, angiotensin II is in vivo converted to angiotensin (1-7) by ACE2, and as a result, homeostasis can be maintained. Therefore, ACE2 can be an essential enzyme in maintaining body homeostasis.

ACE2 is embedded into the cell membrane in the body through the transmembrane site present therein. Due to this property, a great deal of research has recently been reported on the potential of soluble ACE2 (soluble ACE2, sACE2) as an antiviral agent, such as preventing viral infection or inhibiting the proliferation of infected viruses. ACE2 (recombinant soluble ACE2, rsACE2) is also being developed through various genetic recombination. Nevertheless, antiviral drugs using soluble ACE2 have limitations due to low in vivo antiviral efficacy.

Meanwhile, a nanodisc (ND) has a structure in which a phospholipid bilayer is surrounded in the form of a disc with membrane scaffold protein (MSP), which is a protein derived from apolipoprotein A1 (Apo-A1), which is a major ingredient of high-density lipoproteins (HDL) in the body. The nanodisc is mainly used to study the structure of various cell membrane proteins. In addition, the nanodisc (ND) acts as a carrier that delivers various physiological functional substances into the body. Advantageously, the nanodisc is a bio-derived substance and thus is stable in the body, and does not cause harmful reactions and is thus safe.

### DISCLOSURE

### Technical Problem

Accordingly, it is one object of the present invention to provide a method of further improving the antiviral efficacy of conventional angiotensin converting enzyme 2 (ACE2)-based antiviral agents. In addition, it is another object of the present invention to provide a method of further improving the substrate conversion ability of angiotensin converting enzyme 2 (ACE2).

### Technical Solution

In accordance with an aspect of the present invention, the above and other objects can be accomplished by the provision of a nanodisc with improved substrate conversion ability of angiotensin converting enzyme 2 (ACE2) including a lipid bilayer derived from phospholipid, having a flat disc-shaped bilayer structure, and including a hydrophilic moiety disposed outside and a hydrophobic moiety disposed inside, a membrane scaffold protein (MSP) surrounding a side surface of the lipid bilayer where the hydrophobic moiety is exposed to the outside, and angiotensin converting enzyme 2 (ACE2) hydrophobically linked to the inside of the lipid bilayer.

In accordance with another aspect of the present invention, provided is a nanodisc including a lipid bilayer derived from phospholipid, having a flat disc-shaped bilayer structure, and including a hydrophilic moiety disposed outside and a hydrophobic moiety disposed inside, and a membrane scaffold protein (MSP) surrounding a side surface of the lipid bilayer where the hydrophobic moiety is exposed to the outside, wherein the membrane scaffold protein (MSP) is a membrane e scaffold protein fused with angiotensin converting enzyme 2 (ACE2).

Meanwhile, the phospholipid may include at least one selected from the group consisting of phosphatidylcholine, phosphatidylserine, phophatidylethalolamine, phophatidylglycerol, and phophatidylinositol.

The phospholipid may be POPC (1-palmitoyl-2-oleoyl-sn-glycero-3-phosphocholine) or DOPS (1, 2-dioleoyl-sn-glycero-3-phospho-L-serine).

Meanwhile, the membrane scaffold protein may be an amphipathic protein having a helix structure.

Meanwhile, the membrane scaffold protein may be apolipoprotein or a fragment of apolipoprotein that maintains the helix structure and amphipathic characteristics of the apolipoprotein.

Meanwhile, the angiotensin converting enzyme 2 may be soluble angiotensin converting enzyme 2 from which the transmembrane domain has been removed.

Meanwhile, the membrane scaffold protein fused with angiotensin converting enzyme 2 may be produced by binding a gene encoding angiotensin converting enzyme 2 to a gene encoding a membrane structural protein, and expressing the result.

Meanwhile, the nanodisc may improve substrate conversion ability of angiotensin converting enzyme 2 (ACE2).

Meanwhile, the substrate conversion ability may convert angiotensin II into angiotensin 1-7.

### Advantageous effects

The nanodisc of the present invention contains angiotensin converting enzyme 2 (ACE2) and thus exhibits excellent antiviral efficacy and substrate conversion ability.

### DESCRIPTION OF DRAWINGS

FIG. 1 schematically illustrates the structure of a general nanodisc (ND), a nanodisc (NDA) in which angiotensin converting enzyme 2 (ACE2) is bound to a lipid bilayer, and a nanodisc (AND) in which angiotensin converting enzyme 2 (ACE2) is bound to a membrane scaffold protein.

FIG. 2 schematically illustrates a process of producing a nanodisc (NDA) in which angiotensin converting enzyme 2 (ACE2) is bound to a lipid bilayer, and a process of producing a nanodisc (AND) in which angiotensin converting enzyme 2 (ACE2) is bound to a membrane scaffold protein.

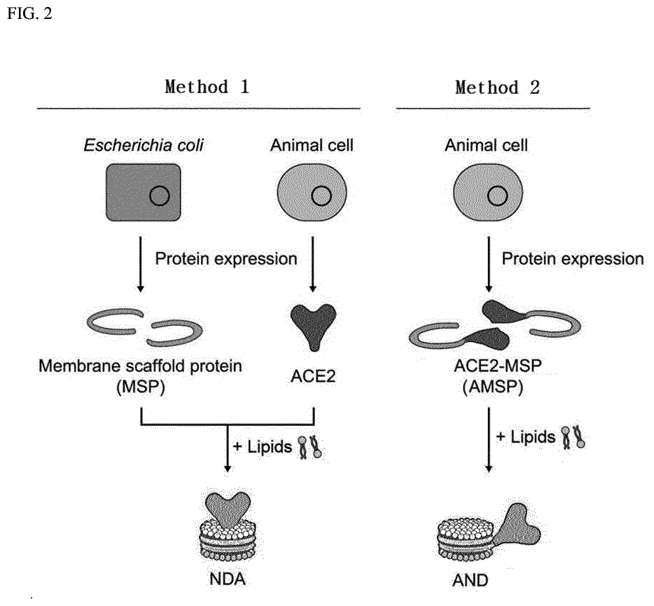

FIG. 3 shows the result of purification using size exclusion chromatography after production of the nanodisc (NDA) in which angiotensin converting enzyme 2 (ACE2) is bound to a lipid bilayer, and the nanodisc (sAND) in which recombinant angiotensin converting enzyme 2 (ACE2) is bound to a membrane scaffold protein.

FIG. 4 shows the results of testing the antiviral efficacy of a mixture of SARS-CoV-2 pseudovirus with recombinant angiotensin converting enzyme 2 (sACE2), a mixture of SARS-CoV-2 pseudovirus with the nanodisc (NDA) in which angiotensin converting enzyme 2 (ACE2) is bound to a lipid bilayer, or a mixture of SARS-CoV-2 pseudovirus with the nanodisc (ND-sACE2) in which recombinant angiotensin converting enzyme 2 (sACE2) is bound to a lipid bilayer.

FIG. 5 shows the result of testing the antiviral efficacy of the nanodisc (NDA) in which angiotensin converting enzyme 2 (ACE2) is bound to a lipid bilayer.

FIG. 6 shows the result of comparison in the antiviral efficacy between nanodiscs (NDA) in which angiotensin converting enzyme 2 (ACE2) is bound to a lipid bilayer, and the nanodisc (sAND) in which recombinant angiotensin converting enzyme 2 (sACE2) is bound to a membrane scaffold protein.

FIG. 7 shows the result of comparison in the substrate conversion ability between recombinant angiotensin converting enzyme 2 (sACE2) and the nanodisc (NDA) in which angiotensin converting enzyme 2 (ACE2) is bound to a lipid bilayer.

### BEST MODE

In one aspect, the present invention is directed to a nanodisc including a lipid bilayer derived from phospholipid, having a flat disc-shaped bilayer structure, and including a hydrophilic moiety disposed outside and a hydrophobic moiety disposed inside, a membrane scaffold protein (MSP) surrounding a side surface of the lipid bilayer, where the hydrophobic moiety is exposed to the outside, and angiotensin converting enzyme 2 (ACE2).

Several coronaviruses, including SARS-CoV and SARS-CoV-2, are known to use angiotensin converting enzyme 2(ACE2) as a receptor to infiltrate human cells. In other words, several coronaviruses, including SARS-CoV and SARS-CoV-2, are known to have the ability to bind to angiotensin converting enzyme 2 (ACE2), and when infected with a coronavirus, angiotensin converting enzyme 2 in the body cannot function properly, causing lung damage and cardiovascular damage. Attempts have been made to develop therapeutic agents for viral infections containing angiotensin converting enzyme 2 (ACE2) based on these properties. However, there is no case that has yet shown excellent efficacy.

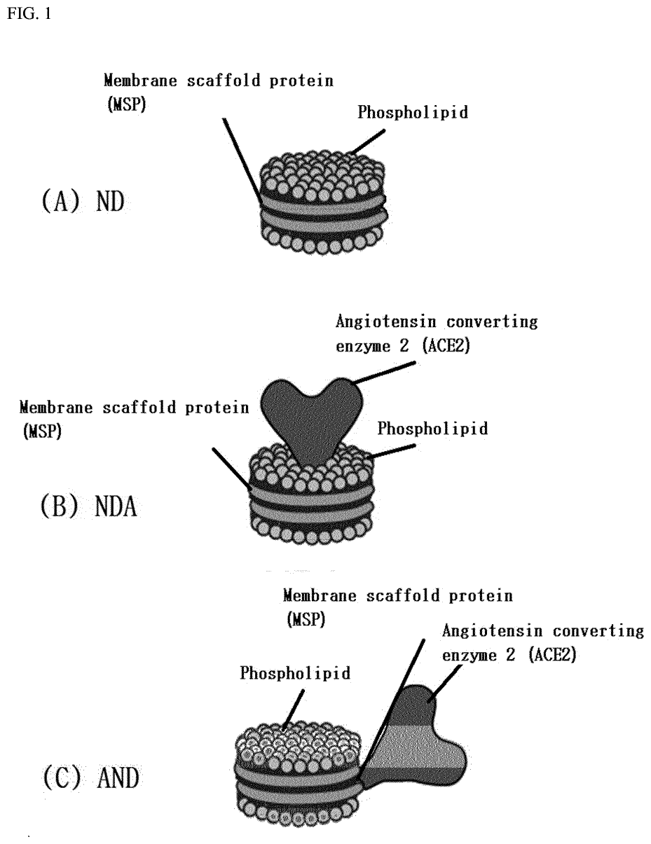

However, in the present invention, when angiotensin converting enzyme 2 (ACE2) was attached to a nanodisc (see FIG. 1), the antiviral efficacy of angiotensin converting enzyme 2 (ACE2) was significantly improved, and the substrate conversion ability of angiotensin converting enzyme 2 (ACE2) was significantly improved. This means that the nanodisc of the present invention can improve the antiviral efficacy of antiviral drugs containing angiotensin converting enzyme 2 (ACE2), or can improve therapeutic effect for ACE2 deficiency and diseases caused thereby of therapeutic agents containing angiotensin converting enzyme 2 (ACE2).

Meanwhile, the lipid bilayer of the present invention has a flat disc-shaped bilayer structure derived from phospholipids and is characterized in that the hydrophilic moiety is disposed outside and the hydrophobic moiety is disposed inside (see FIG. 1).

The lipid bilayer of the present invention is structurally different from a spherical liposome in which either a hydrophobic moiety or a hydrophilic moiety is selectively exposed to the outside in that it has a disc shape in which the hydrophobic moiety as well as the hydrophilic moiety of the amphipathic lipid have a side surface exposed to the outside (see FIG. 1).

Meanwhile, in the present invention, for example, the phospholipid may include at least one selected from the group consisting of phosphatidylcholine, phosphatidylglycerol, phosphatidylethanolamine, phosphatidylserine, and phosphatidylinositol, but is not limited thereto.

The phosphatidylcholine is, for example, DOPC (1,2-dioleoyl-sn-glycero-3-phosphocholine), DLPC (1,2-dilauroyl-sn-glycero-3-phosphocholine), DMPC (1,2-dimyristoyl-sn-glycero-3-phosphocholine), DPPC (1,2-dipalmitoyl-sn-glycero-3-phosphocholine), POPC (1-palmitoyl-2-oleoyl-sn-glycero-3-phosphocholine), C13PC, DDPC (1,2-didecanoyl-sn-glycero-3-phosphocholine), DSPC (1,2-distearoyl-sn-glycero-3-phosphocholine), DEPC (1,2-dierucoyl-sn-glycero-3-phosphocholine), DLOPC (1,2-dilinoleoyl-sn-glycero-3-phosphocholine), EPC (egg phosphatidylcholine), MSPC (1-myristoyl-2-stearoyl-sn-glycero-3-phosphocholine), PMPC (1-palmitoyl-2-myristoyl-sn-glycero-3-phosphocholine), PSPC (1-palmitoyl-2-stearoyl-sn-glycero-3-phosphocholine), SMPC (1-stearoyl-2-myristoyl-sn-glycero-3-phosphocholine) or SPPC (1-stearoyl-2-palmitoyl-sn-glycero-3-phosphocholine)

In addition, the phosphatidylglycerol is, for example, DMPG (1,2-dimyristoyl-sn-glycero-3[phospho-rac-(1-glycerol)], DPPG (1,2-dipalmitoyl-sn-glycero-3[phospho-rac-(1-glycerol)]), DSPG (1,2-distearoyl-sn-glycero-3[phospho-rac-(1-glycerol)), POPG (1-palmitoyl-2-oleoyl-sn-glycero-3[phospho-rac-(1-glycerol)]), DEPG (1,2-dierucoyl-sn-glycero-3[phospho-rac-(1-glycerol)]), DLPG (1,2-dilauroyl-sn-glycero-3[phospho-rac-(1-glycerol)]), DOPG (1,2-dioleoyl-sn-glycero-3[Phospho-rac-(1-glycerol)]) or DSPG (1,2-distearoyl-sn-glycero-3[phospho-rac-(1-glycerol)]). The phosphatidylethanolamine is, for example, DMPE (1,2-dimyristoyl-sn-glycero-3-phosphoethanolamine), DPPE (1,2-dipalmitoyl-sn-glycero-3-phosphoethanolamine), DSPE (1,2-distearoyl-sn-glycero-3-phosphoethanolamine), DOPE (1,2-dioleoyl-sn-glycero-3-phosphoethanolamine), DEPE (1,2-dierucoyl-sn-glycero-3-phosphoethanolamine), DLPE (1,2-dilauroyl-sn-glycero-3-phosphoethanolamine) or POPE (1-palmitoyl-2-oleoyl-sn-glycero-3-phosphoethanolamine), and the phosphatidylserine is, for example, DOPS (1,2-dioleoyl-sn-glycero-3-phosphoserine), DLPS (1, 2-dilauroyl-sn-glycero-3-phosphoserine), DMPS (1, 2-dimyristoyl-sn-glycero-3-phosphoserine); DPPS (1, 2-dipalmitoyl-sn-glycero-3-phosphoserine), DSPS (1,2-distearoyl-sn-glycero-3-phosphoserine) or POPS, and the phosphatidylinositol may be phosphatidylinositol-4-phosphate, phosphatidylinositol-4,5-bisphosphate, or phosphatidylinositol-3,4,5-trisphosphate.

Meanwhile, in the present invention, the membrane scaffold protein (MSP) serves to surround the side surface of the lipid bilayer and for this purpose, it is preferably an amphipathic protein with a helix structure. An example of an amphipathic membrane scaffold protein having a helix structure is apolipoprotein. Apolipoprotein is a protein present specifically in plasma lipoproteins and is known to stabilize the structure of lipoproteins, activate enzymes involved in lipoprotein metabolism, and act as a ligand for lipoprotein receptors present on the cell surface. The apolipoprotein is, for example, apolipoprotein A1 (ApoA-I), apolipoprotein A2 (ApoA-2), apolipoprotein B (ApoB), apolipoprotein C (ApoC), apolipoprotein Protein E (ApoE), MSP1 (membrane scaffold protein 1), MSP1D1, MSP1D2, MSP1E1, MSP1E2, MSP1E3, MSP1E3D1, MSP2, MSP2N1, MSP2N2, MSP2N3, or the like.

ApoA-I, provided as an example above, is known to be an ingredient of high-density lipoprotein (HDL), which plays a direct role in removing cholesterol from surrounding tissues and transporting the same to the liver or other lipoproteins. Apo-A1 is a 28 kDa single polypeptide consisting of 243 amino acids. Apo-A1 is a protein that has 8 repeating unit domains consisting of 11 amino acids or 22 amino acids, and 60 to 75% of alpha-helices in the secondary structure constituting HDL. In addition, like ApoA1, ApoE, is known to be involved in the transport of cholesterol and is a protein composed of a 33 kDa single polypeptide consisting of 299 amino acids.

In addition, in the present invention, a fragment of an apolipoprotein that maintains the helix structure and amphipathic characteristics of the apolipoprotein may be used. In other words, a part (fragment) of the apolipoprotein may be used instead of the entire apolipoprotein as long as the helix structure and amphipathic characteristics of the apolipoprotein are not lost.

Meanwhile, the nanodisc of the present invention is characterized by containing angiotensin converting enzyme 2 (ACE2), which may be bound to a lipid bilayer through hydrophobic interaction. Angiotensin-converting enzyme 2 (ACE2) has a hydrophobic transmembrane domain at the C-terminus, which may bind to the hydrophobic moiety of the lipid bilayer through hydrophobic interaction.

In addition, the angiotensin converting enzyme 2 (ACE2) may be fused to a membrane scaffold protein. Specifically, the nanodisc of the present invention may be produced using a fusion protein prepared by binding a gene encoding angiotensin converting enzyme 2 to a gene encoding the membrane scaffold protein, followed by expressing the result. Meanwhile, according to the examples below, when the fusion protein produced above is used, the production process is simplified and the production yield increases compared to the nanodisc in which angiotensin converting enzyme 2 is bound to the hydrophobic moiety of the lipid bilayer.

Meanwhile, the angiotensin converting enzyme 2 is preferably soluble angiotensin converting enzyme 2 from which the transmembrane domain of angiotensin converting enzyme 2 has been removed.

Angiotensin-converting enzyme 2 (ACE2) is a receptor that is embedded in the cell membrane through a transmembrane domain present therein. Recently, as the ability of angiotensin converting enzyme 2 (ACE2) to bind to viruses has been reported, a great deal of research has been reported on the potential thereof as an antiviral agent to prevent viral infection or inhibit the proliferation of infected viruses. In addition, in order to further improve applicability and functionality, recombinant angiotensin converting enzyme 2 is being developed. In the present invention, soluble angiotensin converting enzyme 2 from which the transmembrane domain has been removed may be used.

Meanwhile, the nanodisc of the present invention has excellent antiviral efficacy. Therefore, the pharmaceutical composition containing the nanodisc of the present invention may be used for preventing or treating viral infections.

In this case, the pharmaceutical composition of the present invention may be used for at least one virus selected from Coronaviridae, Bunyaviridae, Filoviridae, Flaviviridae, Hepadnaviridae, Herpesviridae, Orthomyxoviridae, Poxviridae, Rhabdoviridae, Retroviridae and Togaviridae. Preferably, the pharmaceutical composition is used for viruses that have a spike protein that may bind to angiotensin converting enzyme 2. Specific examples of viruses having the spike protein include SARS-CoV, SARS-CoV-2 and the like.

In addition, the nanodisc of the present invention contains angiotensin converting enzyme 2 and has an excellent substrate conversion ability to convert angiotensin II into angiotensin 1-7. Accordingly, the nanodisc of the present invention may be used to improve, prevent, or treat angiotensin converting enzyme 2 deficiency diseases.

Meanwhile, the pharmaceutical composition according to the present invention may be prepared into a unit dose form, or may be incorporated into a multi-dose container through formulation using a pharmaceutically acceptable carrier and/or excipient according to a method that can be easily implemented by those skilled in the art to which the present invention pertains. Here, the formulation may be prepared in a variety of forms, such as oral medicine or injection, and may be in the form of a solution, suspension, or emulsion in oil or aqueous medium, or may be in the form of an extract, powder, suppository, powder, granule, tablet, or capsule. The composition may further contain a dispersant or a stabilizer.

The pharmaceutical composition of the present invention may be administered orally or parenterally, for example, intrathecally, intravenously, subcutaneously, intradermally, intramuscularly, intraperitoneally, intrasternally, intratumorally, intranasally, intracerebrally, intracranially, intrapulmonarilly, and intrarectally, but is not limited thereto.

Meanwhile, the dosage of the pharmaceutical composition of the present invention may be determined depending on factors such as formulation method, the administration method, the age, weight, gender and pathological conditions of the patient, food, time of administration, route of administration, rate of excretion and responsiveness. An ordinary skilled physician can easily determine and prescribe an effective dosage (pharmaceutically effective amount) for desired treatment or prevention. According to a preferred embodiment of the present invention, the daily dosage of the pharmaceutical composition of the present invention is 0.0001-100 mg/kg.

Hereinafter, the present invention will be described in more detail with reference to the following examples, but the scope of the present invention is not limited to the examples and includes variations and technical concepts equivalent thereto.

### EXAMPLE 1: PREPARATION OF NANODISC OF THE PRESENT INVENTION CONTAINING ANGIOTENSIN CONVERTING ENZYME 2

In this example, the nanodisc of the present invention containing angiotensin converting enzyme 2 was produced.

Meanwhile, the nanodisc of the present invention could be produced using the membrane scaffold protein fused with angiotensin converting enzyme 2 or by binding angiotensin converting enzyme 2 to the lipid bilayer of the nanodisc.

### 1) Purification of Membrane Scaffold Protein Fused with Angiotensin Converting Enzyme 2 (ACE2)

In order to purify membrane scaffold proteins fused with angiotensin converting enzyme 2 (referred to hereinafter as “ACE2”), human embryonic kidney 293 (HEK293) soluble suspended cells were incubated at 37° C. and 120 rpm in the presence of 8% CO₂, to prepare 180 mL (1.1×10⁶ cells/mL) of a cell fluid. Then, 250 μg of plasmid containing the ACE2-MSP1E3D1gene (SEQ ID NO: 1) and 750 μg of PEI (polyethylenimine) were mixed in 20 mL of medium and then mixed with 180 mL of the prepared cell fluid to perform transfection. Then, the transfected cells were incubated in an incubator at 37° C. and 120 rpm in the presence of 8% CO₂ for 72 hours, and then centrifuged at 8,000 g for 10 minutes to obtain the supernatant. The supernatant was treated with Ni-NTA agarose beads, and treated with a buffer containing a low concentration of imidazole (25 mM tris, 150 mM Nacl, 20 mM imidazole, pH 8.0) to remove non-target substances that did not bind to the beads. Then, the residue was treated with a buffer containing a high concentration of imidazole (25 mM tris, 150 mM Nacl, 500 mM imidazole, pH 7.4) to purify proteins fused with membrane scaffold proteins and ACE2 (ACE2-MSP1E3D1, SEQ ID NO: 2).

### 2) Production of Nanodisc (AND) Containing Membrane Scaffold Protein Fused with Angiotensin Converting Enzyme 2 (ACE2)

POPC (1-palmitoyl-2-oleoyl-sn-glycero-3-phosphocholine) and DOPS (1,2-dioleoyl-sn-glycero-3-phospho-L-serine) as lipids were dissolved in chloroform to prepare 25 mg/ml and 10 mg/ml of lipid solutions. Then, 243 μL of the POPC solution and 65 μL of the DOPS solution were transferred to a glass tube, such that the total lipid concentration and volume, when the lipid solutions were dissolved in buffer (40 mM Tris-Cl, 300 mM NaCl, 0.5 mM EDTA, 50 mM NaC, pH 7.4), were adjusted to 10 mM and 1 mL, respectively, and at the same time, the molar ratio of POPC:DOPS was adjusted to 8:2. Then, chloroform was evaporated with nitrogen gas and allowed to stand under vacuum for at least 2 hours to completely remove the solvent to obtain a lipid film. The obtained lipid film was hydrated with 1 mL of the buffer solution and ultrasonicated at 55° C. for 30 minutes to obtain a suspension in which lipids were homogeneously dispersed. The obtained suspension was mixed with the ACE2-MSP1E3D1 purified in step 3) such that the molar ratio of ACE2-MSP1E3D1:lipid was 1:120. Then, the resulting mixture was treated with the same amount of bio-beads as the entire mixture, a total of twice, more specifically, once at room temperature for 5 hours and once at 4° C. for 16 hours, to produce a nanodisc (AND) in which ACE2 was fused to the membrane scaffold protein through a self-assembly process.

### 3) Purification of Membrane Scaffold Protein Fused with Soluble Angiotensin Converting Enzyme 2 (sACE2)

In order to purify membrane scaffold proteins fused with angiotensin converting enzyme 2 (referred to hereinafter as “sACE2”), from which transmembrane domain (SEQ ID NO: 3) has been removed, human embryonic kidney 293 (HEK293) soluble suspended cells were incubated at 37° C. and 120 rpm in the presence of 8% CO₂, to prepare 180 mL (1.1×10⁶ cells/ml) of a cell fluid. Then, 250 μg of plasmid containing the sACE2-MSP1E3D1 gene (SEQ ID NO: 4) and 750 μg of PEI (polyethylenimine) were mixed in 20 mL of medium and then mixed with 180 mL of the cell fluid thus prepared to perform transfection. Then, the transfected cells were incubated in an incubator at 37° C. and 120 rpm in the presence of 8% CO₂ for 72 hours, and then centrifuged at 8,000 g for 10 minutes to obtain the supernatant. The supernatant was treated with Ni-NTA agarose beads, treated with a buffer containing a low concentration of imidazole (25 mM tris, 150 mM Nacl, 20 mM imidazole, pH 8.0) to remove non-target substances that did not bind to the beads. Then, the residue was treated with a buffer containing a high concentration of imidazole (25 mM tris, 150 mM Nacl, 500 mM imidazole, pH 7.4) to purify proteins fused with membrane scaffold proteins and sACE2 (sACE2-MSP1E3D1, SEQ ID NO: 2).

### 2) Production of Nanodisc (sAND) of the Present Invention Containing Membrane Scaffold Protein Fused with Soluble Angiotensin Converting Enzyme 2 (sACE2)

POPC (1-palmitoyl-2-oleoyl-sn-glycero-3-phosphocholine) and DOPS (1,2-dioleoyl-sn-glycero-3-phospho-L-serine) as lipids are dissolved in chloroform to prepare 25 mg/ml and 10 mg/ml of lipid solutions. Then, 243 μL of the POPC solution and 65 μL of the DOPS solution were transferred to a glass tube, such that the total lipid concentration and volume, when the lipid solutions were dissolved in buffer (40 mM Tris-Cl, 300 mM NaCl, 0.5 mM EDTA, 50 mM NaC, pH 7.4), were adjusted to 10 mM and 1 mL, respectively, and at the same time, the molar ratio of POPC:DOPS was adjusted to 8:2. Then, chloroform was evaporated with nitrogen gas and allowed to stand under vacuum for at least 2 hours to completely remove the solvent to obtain a lipid film. The obtained lipid film was hydrated with 1 mL of the buffer solution and ultrasonicated at 55° C. for 30 minutes to obtain a suspension in which lipids were homogeneously dispersed. The obtained suspension was mixed with the sACE2-MSP1E3D1 purified in step 3) such that the molar ratio of sACE2-MSP1E3D1:lipid was 1:120. Then, the resulting mixture was treated with the same amount of bio-e beads as the entire mixture, a total of twice, more specifically, once at room temperature for 5 hours and once at 4° C. for 16 hours, to produce a nanodisc (sAND) in which sACE2 was fused to the membrane scaffold protein through a self-assembly process.

### 5) Purification of Angiotensin Converting Enzyme 2 (ACE2)

In order to purify the protein, human embryonic kidney 293 (HEK293) soluble suspended cells were incubated at 37° C. and 120 rpm in the presence of 8% CO₂, to prepare 180 mL (1.1×10⁶ cells/mL) of a cell fluid. Then, 250 μg of plasmid containing the ACE2 gene (SEQ ID NO: 6) and 750 μg of PEI (polyethylenimine) were mixed in 20 mL of medium and then mixed with 180 mL of the cell fluid thus prepared to perform transfection. Then, the cells were incubated in an incubator at 37° C. and 120 rpm in the presence of 8% CO₂ for 72 hours, and then centrifuged at 8,000 g for 10 minutes to remove the medium and to obtain only the cells. The cells were resuspended using Tris buffer containing 1% DDM, and membrane proteins were separated using an ultra-high-speed centrifuge, and the residue was purified with Ni-NTA agarose beads. The result was treated with a buffer containing 0.1% DDM and low concentration of imidazole to remove substances that did not bind to the beads. Then, the residue was treated with a high concentration of imidazole to obtain ACE2 (SEQ ID NO: 7).

Meanwhile, sACE2 was transfected using a plasmid containing the sACE2 gene (SEQ ID NO: 8) in the same manner as above, and the cells were incubated in an incubator in the presence of 8% CO₂ at 37° C. and 120 rpm for 72 hours, then centrifuged at 8,000×g for 10 minutes to remove the cells, and the supernatant was purified using Ni-NTA agarose beads. Then, substances that did not bind to the beads were removed using a buffer solution containing a low concentration of imidazole, and sACE2 (SEQ ID NO: 9) was obtained using a high concentration of imidazole.

### 6) Production of Nanodisc (NDA) Containing Angiotensin Converting Enzyme 2 (ACE2) Bound with Lipid Bilayer

POPC (1-palmitoyl-2-oleoyl-sn-glycero-3-phosphocholine) and DOPS (1, 2-dioleoyl-sn-glycero-3-phospho-L-serine) as lipids are dissolved in chloroform to prepare 25 mg/ml and 10 mg/ml of lipid solutions. Then, 243 μL of the POPC solution and 65 μL of the DOPS solution were transferred to a glass tube, such that the total lipid concentration and volume, when the lipid solutions were dissolved in buffer (40 mM Tris-C1, 300 mM NaCl, 0.5 mM EDTA, 50 mM NaC, pH 7.4), were adjusted to 10 mM and 1 mL, respectively, and at the same time, the molar ratio of POPC:DOPS was adjusted to 8:2. Then, nitrogen gas was added and the result was allowed to stand under vacuum for at least 4 hours to remove the solvent to obtain a lipid film. The obtained lipid film was hydrated with 1 mL of the buffer solution and ultrasonicated at 55° C. for 30 minutes to obtain a suspension in which lipids were homogeneously dispersed.

Then, ACE2 and MSP1E3D1 were added to the suspension so that the molar ratio of ACE2 (molecular weight of 94.2 kDa) to MSP1E3D1 (molecular weight of 32.6 kDa) to lipid was 0.5:1:120. Then, the result was treated with the same amount of bio-beads as the entire mixture, a total of twice, more specifically, once at room temperature for 5 hours and once at 4° C. for 16 hours, to produce a nanodisc (NDA) in which ACE2 was fused to the lipid bilayer through a self-assembly process.

### 7) Production of Nanodisc (ND-sACE2) Containing Lipid Bilayer Bound with Soluble Angiotensin Converting Enzyme 2 (ACE2)

In order to produce a nanodisc (ND-sACE2) containing lipid bilayer bound with soluble angiotensin converting enzyme 2 (sACE2), POPC, DOP, and 18:1 DGS-NTA (Ni) were transferred to a glass tube such that the total lipid concentration and volume were 10 mM and 1 mL, respectively, and the molar ratio of POPC:DOPS:18:1 DGS-NTA (Ni) was 7:2:1, when they were dissolved in nanodisc (ND) buffer (40 mM Tris-Cl, 300 mM NaCl, 0.5 mM EDTA, 50 mM NaC, pH 7.4). Nitrogen gas was added and the result was allowed to stand under vacuum for at least 4 hours to remove the solvent to obtain a lipid film. The obtained lipid film was hydrated with the NP supplemented with sodium cholate and ultrasonicated at 55° C. for 15 minutes to obtain a suspension containing the lipid film, in which the lipid film was crushed. The obtained suspension was mixed with the membrane scaffold proteins such that the molar ratio of 32.6 kDa MSP1E3D1:lipid was 1:120. Then, the resulting mixture was treated with the same amount of bio-beads (at room temperature for 5 hours), to produce a nanodisc (AND) through a self-assembly process. The nanodisc produced using the method was mixed with sACE2 at 4° C. for 1 hour to produce a nanodisc (ND-sACE2) in which soluble angiotensin converting enzyme 2 (sACE2) was bound to the lipid bilayer. Meanwhile, sACE2 is covalently linked to the His-tag at the C terminus of 18:1 DGS-NTA (Ni) contained in the nanodisc.

### 8) Purification of Nanodisc of the Present Invention

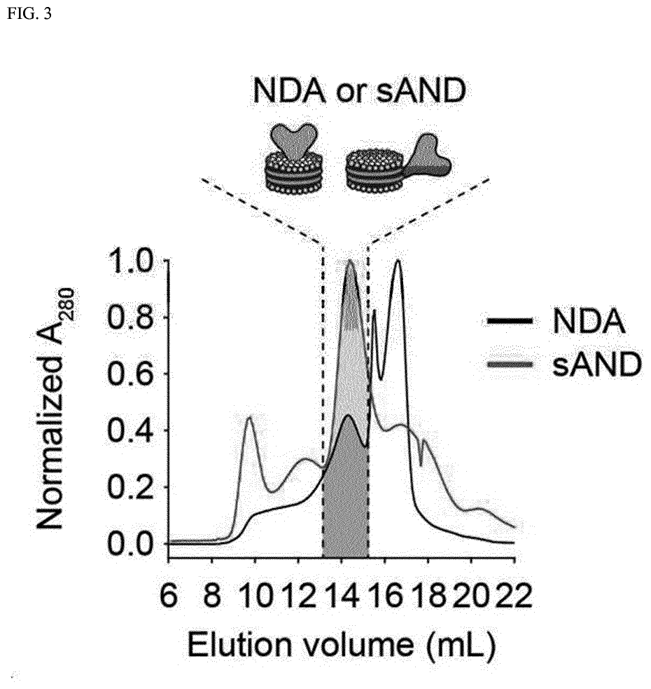

The nanodisc (sAND) of the present invention produced in step 4) and the nanodisc (NDA) produced in step 6) were purified through size exclusion chromatography (SEC). As a result, nanodiscs (NDA or sAND having molecular weight of about 400 kDa) containing ACE2 could be purified at an elution volume of 13 to 15 mL (FIG. 3).

Then, the final production yield was obtained by dividing the amount of protein used to produce the nanodisc by the amount of protein contained in the final purified nanodisc and converting the result into a percentage. The nanodisc (SAND) in which soluble angiotensin converting enzyme 2 was fused to the membrane scaffold protein had a yield of 47.0%, whereas the nanodisc (NDA) in which angiotensin converting enzyme 2 was fused to a lipid bilayer had a yield of 26.2%.

### EXAMPLE 2: CONFIRMATION OF SUBSTRATE CONVERSION ABILITY AND ANTIVIRAL EFFICACY OF NANODISC OF THE PRESENT INVENTION

In this example, the excellent substrate conversion ability and antiviral efficacy of the nanodisc of the present invention were confirmed.

### 1) Confirmation of Antiviral Efficacy of Nanodisc (NDA or ND-sACE2) in which ACE2 or sACE2 is Bound to the Lipid Bilayer

The virus inhibition ability was evaluated by quantifying the degree of infection with SARS-CoV-2 pseudovirus (PV) in “HEK293-ACE2”, which is a cell that expressed ACE2 in 293T cells grown in DMEM supplemented with 5% FBS, or “HEK293-ACE2/TMPRSS2”, which is a cell that expressed simultaneously ACE2 and TMPRSS2 (transmembrane protease serine subtype 2, SEQ ID NO: 10, derived from humans) in 293T cells grown in DMEM supplemented with 5% FBS.

It is known that SARS-CoV-2 infects cells through two main routes. The first is endocytosis and the second is direct fusion. The first route occurs when the cell expresses only ACE2 on the surface and the second route occurs when the cell simultaneously expresses ACE2 and TMPRSS2. Therefore, for the experiments in the two routes, “HEK293-ACE2”, which expressed ACE2 in HEK-293T cells, and “HEK293-ACE2/TMPRSS2”, which expressed ACE2 and TMPRSS2 simultaneously in HEK-293T cells, were used.

Specifically, 100 μL of the two cells were seeded at a concentration of 2×10⁵ cell/mL into each well of a white 96-well cell culture plate and incubated in a 5% CO₂ incubator at 37° C. for 24 hours. After 24 hours, SARS-CoV-2 pseudovirus was first prepared at a concentration of 50 TCID50.

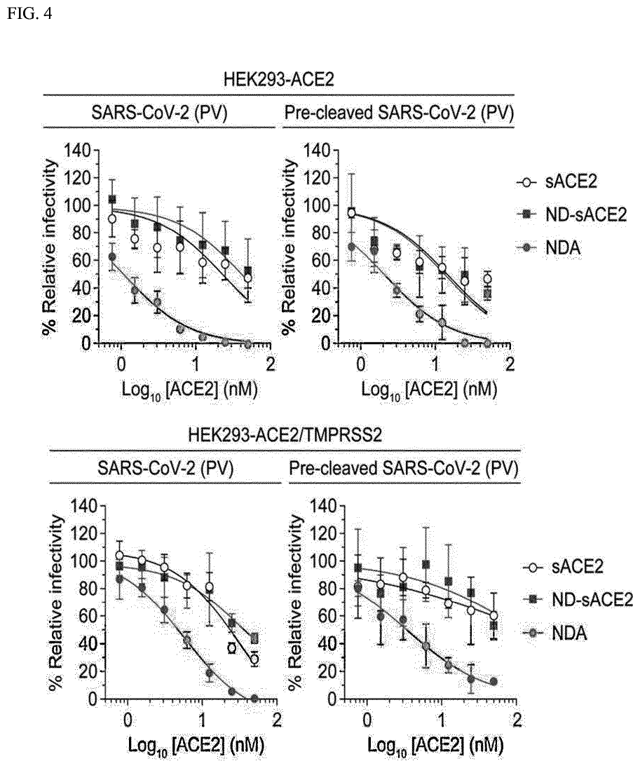

The SARS-CoV-2 pseudovirus was reacted with soluble angiotensin converting enzyme 2 (sACE2) purified in 5) of Example 1, or the nanodisc (NDA or ND-sACE2) containing a lipid bilayer bound to ACE2 or sACE2 prepared in 6) or 7) of Example 1 at 37° C. for 1 hour. Then, the medium was removed from the cells, and 100 μL of the virus and antiviral agent mixture was seeded into each well containing the cells and incubated in a 5% CO₂ incubator at 37° C. for 72 hours. Then, the virus inhibition ability of the antiviral agent was measured using a Luciferase assay kit (FIG. 4).

In addition, in FIG. 4, antiviral activity of the pre-cleavage experimental group (an experimental group treated with trypsin to increase the infectivity of the virus in infection through direct fusion) was measured. It was reported that infection based on direct fusion (HEK293-ACE2/TMPRSS2) exhibits increased cell infection ability, when the spike protein, the protruding protein of SARS-CoV-2, is pre-cleaved, compared to the case where the spike protein is not cleaved (non-cleavage experimental group). In other words, if the virus is pre-cleaved through trypsin treatment, the virus infection power becomes stronger. In this experiment, an experimental group with increased infectious ability of the virus was established and the antiviral activity thereof was measured. Specifically, SARS-CoV-2 pseudovirus was treated with 5 μg/mL of TPCK-trypsin at 37° C. for 1 hour, and then 5% FBS was added thereto to stop the reaction. Then, the virus-inhibiting ability of the antiviral agent was measured using the same method as above (FIG. 4).

The result of the experiment showed that the nanodisc (NDA), in which ACE2 was bound to the lipid bilayer, had excellent virus inhibition ability in all experimental groups. This means that the nanodisc (NDA), in which ACE2 is bound to a lipid bilayer, binds more stably and strongly to the viral spike protein and thus exhibits better antiviral efficacy, than sACE2.

On the other hand, the nanodisc (ND-sACE2) in which SACE2 was bound to a lipid bilayer exhibited antiviral efficacy at a level similar to that of sACE2.

### 2) Confirmation of Antiviral Efficacy of Nanodisc (NDA) in which ACE2 is Bound to Lipid Bilayer Against Virus Variants

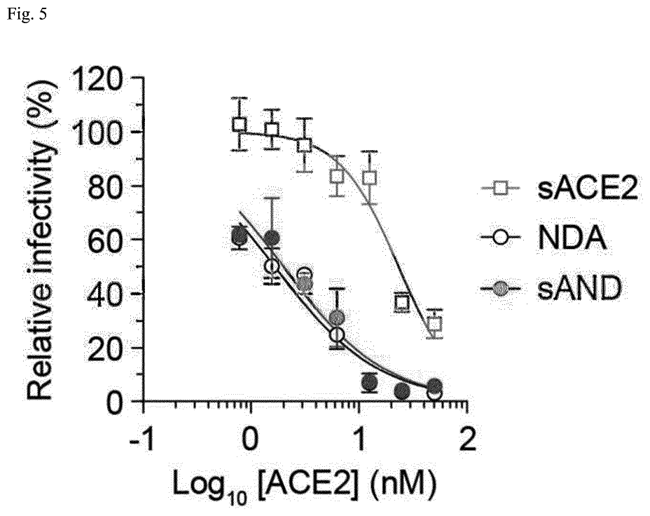

An experiment was performed using the method of 1) above based on a non-cleavage test method, by applying HEK293-ACE2/TMPRSS2 to pseudoviruses (PV) such as ARS-CoV-1, SARS-CoV-2, SARS-CoV-2 Delta, Omicron BA.1, Omicron BA.2, Omicron BA.4, and Omicron BA.5. Meanwhile, the experiment was performed using, as a control group, P2B-2F6, which an antibody showing efficacy against coronavirus, (Ju, B. et al. Human neutralizing antibodies elicited by SARS-CoV-2 infection. Nature 584, 115-119 (2020)), CR3002 (Ter Meulen, J. et al. Human monoclonal antibody combination against SARS coronavirus: synergy and coverage of escape mutants. PLoS Med. 3, e237 (2006)), REGN10933 (Hansen, J. et al. Studies in humanized mice and convalescent humans yield a SARS-CoV-2 antibody cocktail. Science 369, 1010-1014 (2020)) (FIG. 5).

The result of the experiment showed that the nanodisc (NDA) in which ACE2 is bound to the lipid bilayer exhibited better virus inhibition ability than antibodies (P2B-2F6, CR3022, ERGN10933) in all experimental groups. In addition, it can be seen that the nanodisc (NDA), in which ACE2 is bound to the lipid bilayer, exhibits excellent efficacy against all coronavirus variants. However, when antibodies (P2B-2F6, CR3022, ERGN10933) are used, they exhibit efficacy only on some coronavirus variants. This means that, compared to other types of drugs, drugs containing nanodiscs (NDAs) in which ACE2 is bound to a lipid bilayer can effectively respond regardless of the mutation of the virus.

### 3) Comparison in Antiviral Efficacy between Nanodisc (NDA), in which ACE2 is Fused to a Lipid Bilayer, and Nanodisc (SAND), in which sACE2 is Fused to a Membrane Scaffold Protein

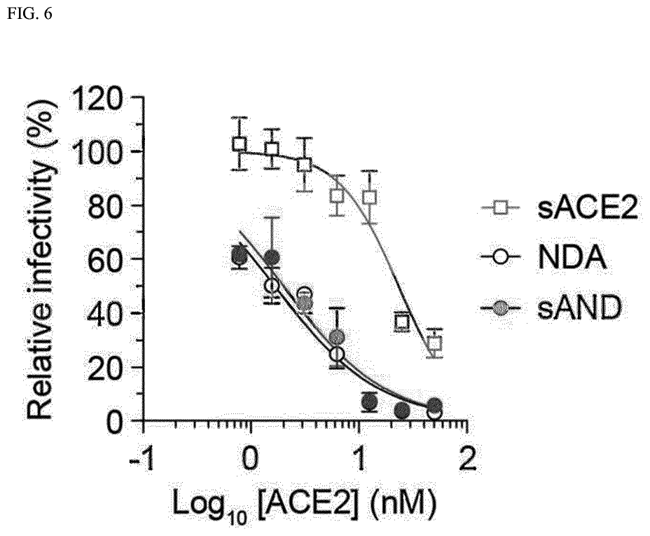

An experiment was performed on the nanodisc in which ACE2 is bound to a lipid bilayer (NDA) and the nanodisc in which sACE2 is fused to a membrane scaffold protein (sAND) using the method of 1) above based on a non-cleavage test method, by applying HEK293-ACE2/TMPRSS2 to pseudoviruses (PV) (FIG. 6).

The result of the experiment showed that the nanodisc (SAND), in which sACE2 is fused to a membrane scaffold protein, also exhibits antiviral efficacy comparable to the nanodisc (NDA), in which ACE2 is fused to a lipid bilayer.

### 4) Confirmation of Substrate Conversion Ability of Nanodisc (NDA) in which ACE2 is Bound to Lipid Bilayer

The ACE2 activity assay kit was used to evaluate the substrate conversion ability of the nanodisc (NDA) in which ACE2 is bound to the lipid bilayer. When an MCA-based peptide substrate is added, ACE2 has the ability to cleave the MCA-based peptide substrate to release a free fluorophore, and the ability is measured using a fluorescence microplate reader to determine the substrate conversion ability of sACE2 produced in Example 1-5) and the nanodisc (NDA) produced in Example 1-6).

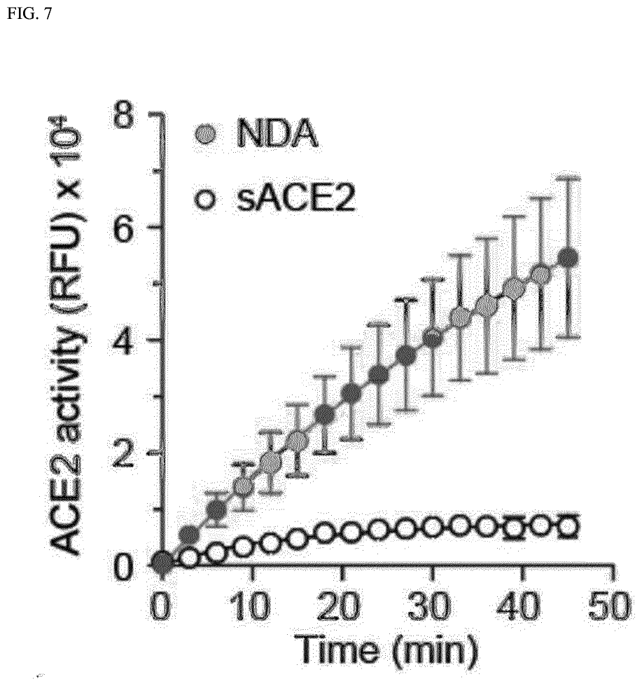

Specifically, 50 μL of sACE2-and ACE2-containing nanodiscs (NDA) were seeded into each well such that the concentration of sACE2 was 0.125 μg/mL, and 50 μL of MCA substrate was added to each well, and the substrate conversion ability was measured at an absorption wavelength of 320 nm/emission wavelength of 420 nm (FIG. 7).

This indicates that ACE2 exhibits better substrate conversion ability of the nanodisc (NDA) bound to a lipid bilayer than sACE2, which means that the nanodisc stabilizes ACE2 so that ACE2 can perform the original enzyme function thereof more effectively than sACE2.

1. A nanodisc with improved substrate conversion ability of angiotensin converting enzyme 2 (ACE2) comprising:
a lipid bilayer derived from phospholipid, having a flat disc-shaped bilayer structure, and including a hydrophilic moiety disposed outside and a hydrophobic moiety disposed inside;
a membrane scaffold protein (MSP) surrounding a side surface of the lipid bilayer where the hydrophobic moiety is exposed to the outside; and
angiotensin converting enzyme 2 (ACE2) hydrophobically linked to the inside of the lipid bilayer.

2. A nanodisc comprising:
a lipid bilayer derived from phospholipid, having a flat disc-shaped bilayer structure, and including a hydrophilic moiety disposed outside and a hydrophobic moiety disposed inside; and
a membrane scaffold protein (MSP) surrounding a side surface of the lipid bilayer where the hydrophobic moiety is exposed to the outside,
wherein the membrane scaffold protein (MSP) is a membrane scaffold protein fused with angiotensin converting enzyme 2 (ACE2).

3. The nanodisc according to claim 1, wherein the phospholipid comprises at least one selected from the group consisting of phosphatidylcholine, phosphatidylserine, phophatidylethalolamine, phophatidylglycerol, and phophatidylinositol.

4. The nanodisc according to claim 1, wherein the phospholipid is POPC (1-palmitoyl-2-oleoyl-sn-glycero-3-phosphocholine) or DOPS (1,2-dioleoyl-sn-glycero-3-phospho-L-serine).

5. The nanodisc according to claim 1, wherein the membrane scaffold protein is an amphipathic protein having a helix structure.

6. The nanodisc according to claim 1, wherein the membrane scaffold protein is apolipoprotein or a fragment of apolipoprotein that maintains the helix structure and amphipathic characteristics of the apolipoprotein.

7. The nanodisc according to claim 1, wherein the angiotensin converting enzyme 2 is soluble angiotensin converting enzyme 2 from which the transmembrane domain has been removed.

8. The nanodisc according to claim 2, wherein the membrane scaffold protein fused with angiotensin converting enzyme 2 is produced by binding a gene encoding angiotensin converting enzyme 2 to a gene encoding a membrane structural protein, and expressing the result.

9. The nanodisc according to claim 2, wherein the nanodisc improves substrate conversion ability of angiotensin converting enzyme 2 (ACE2).

10. The nanodisc according to claim 1, wherein the substrate conversion ability converts angiotensin II into angiotensin 1-7.

11. The nanodisc according to claim 2, wherein the phospholipid comprises at least one selected from the group consisting of phosphatidylcholine, phosphatidylserine, phophatidylethalolamine, phophatidylglycerol, and phophatidylinositol.

12. The nanodisc according to claim 2, wherein the phospholipid is POPC (1-palmitoyl-2-oleoyl-sn-glycero-3-phosphocholine) or DOPS (1,2-dioleoyl-sn-glycero-3-phospho-L-serine).

13. The nanodisc according to claim 2, wherein the membrane scaffold protein is an amphipathic protein having a helix structure.

14. The nanodisc according to claim 2, wherein the membrane scaffold protein is apolipoprotein or a fragment of apolipoprotein that maintains the helix structure and amphipathic characteristics of the apolipoprotein.

15. The nanodisc according to claim 2, wherein the angiotensin converting enzyme 2 is soluble angiotensin converting enzyme 2 from which the transmembrane domain has been removed.

16. The nanodisc according to claim 9, wherein the substrate conversion ability converts angiotensin II into angiotensin 1-7.
